(19) United States
(12) Patent Application Publication
(10) Pub. No.: US 2015/0323533 A1
(43) Pub. Date: Nov. 12, 2015

(54) Detection Device for In Vivo and/or In Vitro Enrichment of Sample Material

(71) Applicant: GILUPI GmbH, Potsdam (DE)

(72) Inventors: Stephan Sass, Potsdam (DE); Dörte Steinbrück, Potsdam (DE); Hans-Gerd Löhmannsröben, Wolfsburg (DE); Solveigh Krusekopf, Berlin (DE); Klaus Lücke, Potsdam (DE); Robert Niestroj-Pahl, Potsdam (DE); Andrè Gessner, Potsdam (DE); Anne Techen, Potsdam (DE); Frank Morgner, Schwielowsee (DE)

(21) Appl. No.: 14/707,542

(22) Filed: May 8, 2015

(30) Foreign Application Priority Data
May 9, 2014 (DE) 10 2014 006 906.0

(51) Int. Cl. G01N 33/569

(52) U.S. Cl. CPC G01N 33/56966; G01N 2333/70596

(57) ABSTRACT

The invention relates to a detection device for in vivo and/or in vitro enrichment of sample material, including a functional surface equipped with detection receptors, in which the functional surface is located directly or indirectly on a measuring structure that includes optically conductive material for optical measurement of target molecules enriched on the detection receptors.

### CROSS REFERENCE TO RELATED APPLICATIONS

This application claims priority to German Patent Application No. 10 2014 006 906.0 filed May 9, 2014, the disclosure of which is hereby incorporated by reference in its entirety.

### BACKGROUND OF THE INVENTION

1. Field of the Invention

The invention relates to a detection device for in vivo and/or in vitro enrichment of sample material, comprising a functional surface equipped with detection receptors.

2. Description of Related Art

Although many cell types, molecules, tumor markers and biomarkers are present in body liquids, it is often not possible to obtain them—on account of their low concentration—in an adequately efficient way with the conventional enrichment methods so as to use them later in established diagnostic methods of clinical chemistry, pathology and cytology.

For instance, it is possible to enrich special cells, particularly circulating tumor cells, from a blood sample (in vitro) by use of commercially available paramagnetic nanoparticles and/or by density gradient centrifugation, but only in a very limited number and with the drawback that the nanoparticles bind to or in the cell and might thus damage them or complicate diagnosis. One of these commercial methods lies in a test in which e.g. circulating tumor cells from 7.5 ml blood volume are enriched by means of paramagnetic nanoparticles to be then able to make statements on the progress of the disease.

The restrictive factor of this method is the obtained sample volume, which is many times greater when a detection device is used for the in vivo enrichment of sample material, e.g. a functionalized catheter. Vascular catheters for the application of medical interventions have a cylindrical design most of the time. Such a detection device is known, for example, from WO 2010/145824 A1.

### SUMMARY OF THE INVENTION

In some examples, there is provided a detection device for the in vivo and/or in vitro enrichment of sample material, comprising a functional surface equipped with detection receptors, wherein said functional surface is arranged directly or indirectly on a measuring structure comprising optically conductive material for optical measurement of target molecules enriched on said detection receptors.

Additionally provided are uses for a detection device. Particularly, use of a detection device for the invasive enrichment of sample material, in particular for the enrichment of cells, comprising preferably circulating endothelial cells or disseminated tumor cells, in particular of hematogenically metastasizing tumors; for the elimination of drugs; for the elimination of radioactive tracers; for the elimination of magnetic beads; for obtaining tumor markers or biomarkers; and/or for the elimination of toxins.

Also, methods for the enrichment of sample material, are provided. Methods for enrichment comprising: providing a detection device exposing functional surface of said detection device to a sample liquid; enriching sample material, preferably cells, DNA, RNA, proteins, peptides, synthetic molecules by exposing said sample material to the detection receptors of said detection device; marking said sample material enriched on said detection receptors, preferably with a fluorescently marked antibody; and optically detecting said sample material by said measuring structure by use of optic measurement, preferably fluorescence measurement.

### BRIEF DESCRIPTION OF THE DRAWINGS

The foregoing summary, as well as the detailed description, will be better understood when read in conjunction with the appended drawings. The present invention is described herein in greater detail using an embodiment and associated drawings. In the drawings:

FIG. 1 is a schematic illustration of a detection device with a measuring structure prior to the formation of a functional surface;

FIG. 2 is a schematic illustration of a detection device according to the invention with a measuring structure and a functional surface on an applied functional layer made of a polymer;

FIG. 3 schematically shows the binding of target cells of blood by use of a detection device according to the invention; and

FIG. 4 schematically shows the marking of bound target cells as well as their optical detection by use of the detection device according to the invention.

### DETAILED DESCRIPTION

The invention is based on the object to provide a detection device by use of which the quantity and/or the specificity of sample material obtained can be determined with less effort.

To satisfy the object underlying the present invention, a detection device is provided for the in vivo and/or in vitro enrichment of sample material, the detection device comprising a functional surface equipped with detection receptors, wherein the functional surface is located directly or indirectly on a measuring structure that comprises optically conductive material for optical measurement of target molecules enriched at the detection receptors.

The detection device according to the invention thereby enables optically measuring the obtained sample material in a time-saving manner and with little effort, in particular by employing fluorescence measurement, and thereby to determine the number and/or the specificity of the target molecules or target cells obtained by the detection receptors. Time-consuming counting and/or identification of target cells obtained can thereby be dispensed with or be shortened. With only little effort can it therefore be determined whether the respective target cells have been bound in sufficient number and that sample taking was therefore successful, or whether further target cells are required for a sufficiently reliable diagnosis.

The detection device according to the invention also ensures that specific target cells or target molecules, respectively, are by optical measurement, in particular fluorescence measurement, distinguished from non-specific cells. This can be accomplished, for example, in that after sample taking, specific target cells and nonspecific cells are marked differently so that a distinction is possible by fluorescence measurement. After sample taking and optical measurement, the sample material can then be made available to the respectively required diagnostic procedures.

The functional surface can be equipped with chemically identical or chemically different detection receptors. Different ligands or target cells, respectively, can therefore be enriched in an application as needed. Within the meaning of this invention all structures, in particular receptors or ligands, which are suited for capturing target molecules and target cells, are referred to as detection receptors. Furthermore, all target molecules and target cells that can dock to the detection receptors are in a simplifying manner referred to as ligands. The term sample liquid refers to a sample given in liquid form.

In the following, “proximal” is further understood to be the side facing the respective operator of the detection device, where “distal” is understood to be the side facing the respective patient.

Preferred embodiments of the invention are the subject matter of the dependent claims.

It can prove to be useful when the detection device according to the invention, in particular its function surface, is formed according to the device described in WO 2010/145824 A1, with the proviso that a measuring structure according to the invention is provided. The disclosure content of WO 2010/145824 A1 is to be part of the present patent application, and is incorporated herein by reference in its entirety.

It may furthermore be advantageous if the measuring structure comprises an optical waveguide made of plastic material and/or that the measuring structure comprises an optical fiber, preferably a polymeric optical fiber. The optical fiber can in particular be made of non-fluorescent material. For example, the optical fiber can be made of CYTOP® amorphous fluoropolymer, FONTEX® fluororesin-based plastic optical fiber, or PMMA (Poly(methyl methacrylate)).

According to an advantageous embodiment of the detection device, the measuring structure can comprise a single or a plurality of interconnected and/or twisted fibers. The measuring structure can just as well form a continuous core. It is also possible that the measuring structure is formed as a carrier for the functional surface. Finally, the measuring structure can additionally be formed as a separate carrier for the functional surface, for example, in addition to a metal guide wire. Preferably, however, a conventional guide wire is replaced entirely by a measuring structure formed as a guide element enabling particularly accurate measurement of target molecules while at the same time having a small number of device components.

It can further be helpful if the measuring structure at its proximal end has a connection for an optical measuring instrument, preferably for a fiber-coupled spectrometer or a photodiode unit. It can also be advantageous if the measuring structure is at its proximal end section-wise enclosed by a holder.

According to yet another advantageous embodiment of the detection device, the detection receptors comprise antibodies, antibody fragments, amino acid structures, nucleic acid structures and/or synthetic structures with a specific affinity to the surfaces of target cells, preferably monoclonal antibodies of murine origin, chimeric antibodies or humanized antibodies, preferably anti-CD146 and/or anti-EpCAM antibodies. Furthermore, the detection receptors are preferably usable for the detection of analytes in body liquids and/or rare cells in body liquids, preferably endothelial cells circulating in the blood.

The detection device can in a further advantageous embodiment be characterized in that the functional surface extends over a functional section formed at the distal end of the detection device, where the functional section preferably has a length of 20-60 mm and/or a thickness of 0.1 to 1.0 mm, particularly preferably of 0.5 mm. The functional section can there particularly preferably be inserted into a vein or other vessels or body cavities.

It can finally be helpful if the functional surface equipped with the detection receptors is formed by a functional layer of blood-repelling and/or biocompatible, and/or hemocompatible material disposed along the functional section of the detection device directly or indirectly on the measuring structure, where the hemocompatible material preferably consists of a natural biopolymer, is particularly preferably made of alginate and/or has hydrophilic properties and/or is formed as a hydrogel.

According to another embodiment of the detection device, the functional surface can section-wise or entirely be equipped with detection receptors. The functional surface can also be equipped with detection receptors of different kinds or with detection receptors having different specificities.

In yet a further advantageous embodiment, the target molecules enriched at the detection receptors can be marked by use of an antagonist, preferably by use of a fluorescently marked antibody. It can also be advantageous if target molecules enriched at the detection receptors can by use of the measuring structure be detectable by fluorescence spectroscopy by determining a wavelength shift and/or a change in intensity and/or a change in the luminescence decay.

The functional surface can in one advantageous embodiment be coated with a protective layer, where the protective layer is preferably soluble in liquids, in particular in body liquids, preferably in blood and/or is biocompatible.

The detection receptors can be operatively connected preferably via linkers or organic functional groups to the functional surface, preferably to the measuring structure or the functional layer, preferably by chemical bonding, particularly preferably by covalent binding. The detection receptors can in particular be coupled via carboxyl groups to the functional surface, where coupling of the detection receptors is effected preferably via carbodiimide chemistry and/or UV cross-linking and/or where the carboxyl groups can be provided at the measuring structure or within the functional layer.

The detection receptors can be subsequently coupled to a functional surface. It is also possible to apply the detection receptors together with a functional layer, for example, made of a biocompatible polymer, onto the measuring structure, the functional layer thus applied forming the functional surface.

The functional layer can be operatively connected to the measuring structure by use of a bonding agent. Such a bonding agent can comprise, for example, amino groups and/or carboxyl groups. The functional layer can just as well be operatively connected to the measuring structure without the use of a bonding agent.

Another independent aspect of the invention relates to a detection device according to at least one of the preceding embodiments, produced in that a measuring structure with a functional section is first provided and then functionalization of the functional section with detection receptors is performed for forming a functional layer, where coupling the detection receptors is preferably done by carbodiimide chemistry.

A detection device according to the invention can in a preferable manner be used for the invasive enrichment of sample material, in particular for enrichment of cells, comprising preferably circulating endothelial cells or disseminated tumor cells, in particular of hematogenically metastasizing tumors, or for the elimination of drugs, for the elimination of radioactive tracers, for the elimination of magnetic beads, for obtaining tumor markers or biomarkers and/or for the elimination of toxins.

Yet another independent aspect of the invention refers to a method for the enrichment of sample material by using a detection device according to at least one of the preceding embodiments, the method comprising the following steps:
a. exposing the functional surface to a sample liquid,
b. enriching sample material, preferably cells, DNA, RNA, proteins, peptides, synthetic molecules on the detection receptors,
c. marking the sample material enriched on the detection receptors, preferably with a fluorescently marked antibody,
d. optically detecting the sample material by the measuring structure by use of optic measurement, preferably fluorescence measurement.

The present invention is described below in greater detail using an embodiment and associated drawings.

The detection device 1 of the invention according to the embodiment illustrated is a biofunctionalized medical detection catheter for invasive (in vivo) enrichment of rare cells, biomolecules, or drugs. Such a detection catheter is also referred to as a medical nano-catheter (MN-C).

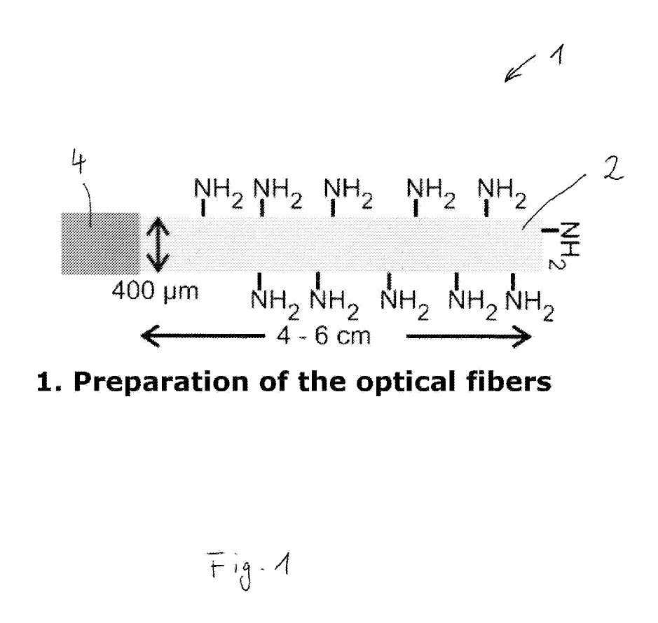

FIG. 1 shows a schematically illustrates the detection catheter 1 prior to the application of a functional layer. Detection device 1 according to FIG. 1 comprises a measuring structure configured as a carrier 2. Measuring structure 2 can be formed by one or several optical fibers. The functionalizable section of measuring structure 2 can, for example, have a length of 4 to 6 cm. The entirety of the optical fibers can have a diameter of about 400 μm. A single fiber can also be provided with a diameter of about 400 μm. Measuring structure 2 can at the proximal end of detection device 1 be enclosed by a holder 4.

A bonding agent can be provided on the surface of measuring structure 2 and can comprise, for example, amino groups and/or carboxyl groups. In FIG. 1, the bonding agent is indicated schematically by the designation NH₂ (amino groups). It is also without a bonding agent possible to achieve bonding of a functional layer on the measuring structure 2.

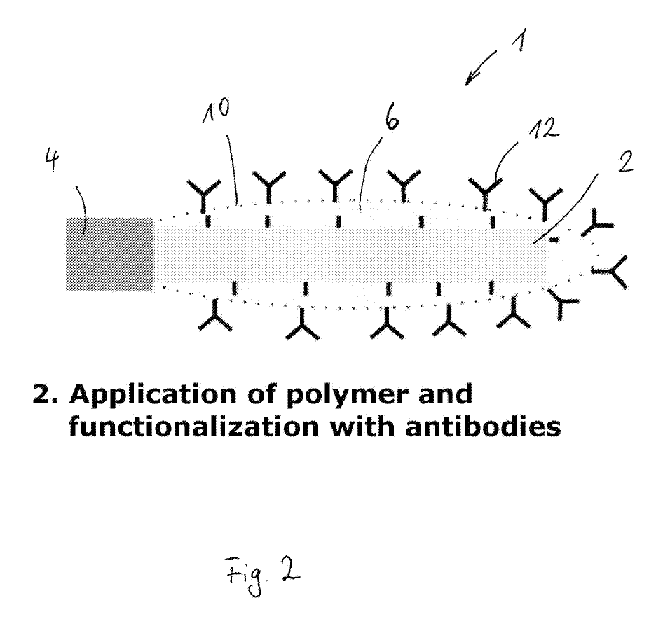

FIG. 2 shows an embodiment of detection device 1 after the application of a functional layer 6. Functional layer 6 can, for example, be made of a polymer. A functional surface 10 is formed on functional layer 6 and is equipped with detection receptors 12. However, functional surface 10 may also be formed directly on measuring structure 2.

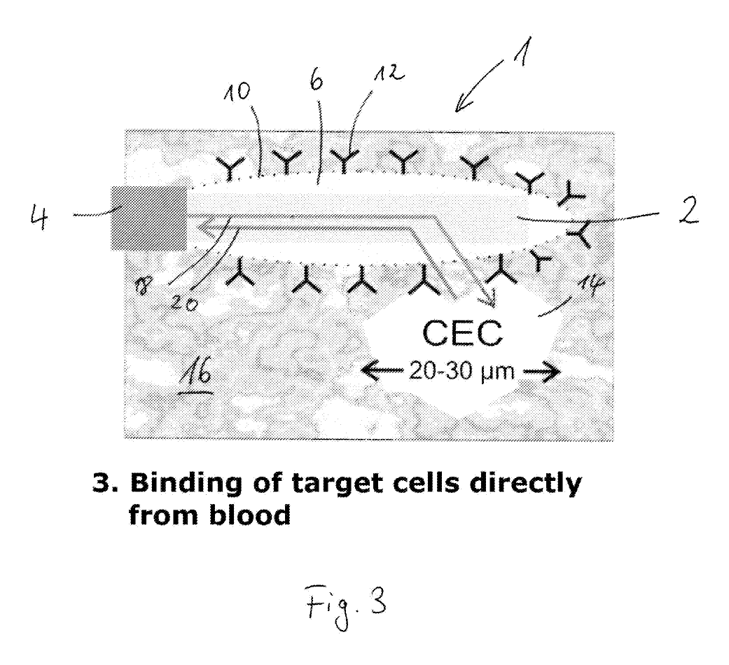

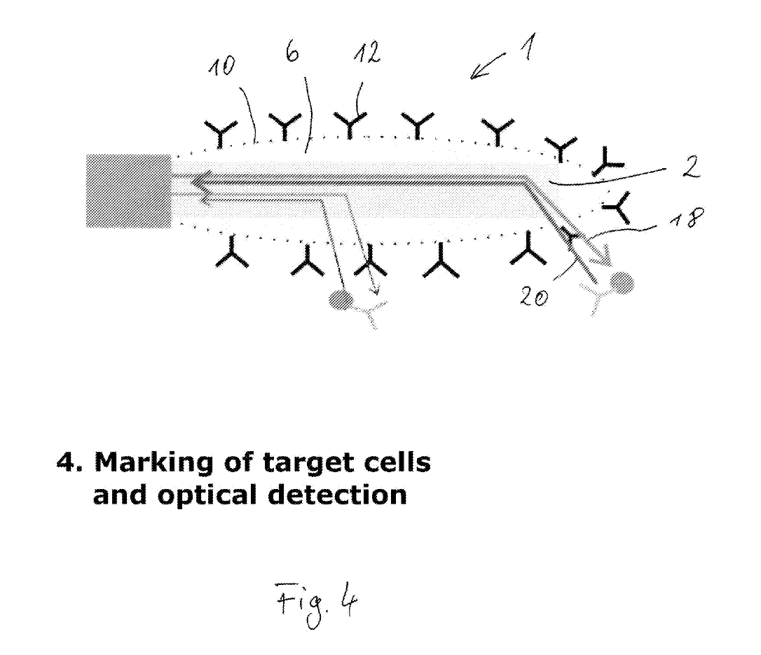

It is in FIG. 3 schematically shown that target molecules 14 of blood 16 are bound by detection receptors 12. It is with arrows 18 and 20 indicated that bound target molecules 14 can be optically measured by measuring structure 2. It is in particular possible to determine in this manner the number of bound target molecules or target cells, respectively. As shown in FIG. 4, it can be necessary for optical measurement, in particular for fluorescence measurement, to color the bound target molecules or target cells, respectively, and to then perform the optical detection.

As described above, functional layer 6 can be made of a polymer, in particular, of a biocompatible polymer. The biocompatible polymer is preferably a hydrogel with carbon-containing long branched macromolecules which have a great number of functional groups, e.g. carboxyl groups or polycarboxylate groups, respectively. The type of functional groups depends on the molecular properties of specific detection receptors 12. The biocompatible hydrogel thereby ensures the permanent covalent binding of detection receptors 12 while maintaining the biological function and simultaneously prevents impairment of the detection function of detection receptors 12 by non-specific adsorption phenomena. Hydrogels are three-dimensionally crosslinked hydrophilic polymers that absorb liquids such as water, but are themselves not soluble therein. Main constituents of the hydrogel are polyacrylic acid (PAA) and polyethylene glycol (PEG).

Property profiles can be customized in response to the desired requirements or fields of application through an appropriate selection of the monomer units, the crosslinking degree and the crosslinking density. An essential property is biocompatibility, i.e. compatibility of the hydrogel with the living tissue. Due to the branched polymer chains of biocompatible polymer 3 the thrombogenic effect during invasive application is also suppressed. Due to chemical activation the functional groups receive an unbalanced molecule charge that makes it possible to electrostatically attract detection receptors 12 from a solution and to covalently bind them. Detection receptors 12 which are permanently immobilized on polymer layer 3 serve the specific binding of the ligands or the target molecules and target cells through their surface antigens, thereby enabling the function of detection device 1. This biocompatible polymer can additionally contain chemically or enzymatically cleavable groups to simplify the quantitative recovery of bound target molecules or cells.

The branched molecular structures of biocompatible polymer 6 can form a functional surface 10 which is three-dimensionally structured in the microscopic and/or the visible range and includes mutually facing functional sections and spaces which can be filled with sample liquid.

For preserving and for protection against the conditions of final sterilization and for radiation protection and for the stability of the product, a biocompatible protective layer (tertiary layer or stabilizer layer) can be applied over the biocompatible polymer—presently not shown. This protective layer dries up over the functional layer and forms a dense network of crystalline structures and stabilizes and thereby preserves the functional part of detection device 1. The protective layer is not covalently bound. The protective layer dissolves in the bloodstream and exposes functional surface 10 of the catheter. Alternatively, the protective layer can be washed off with sterile water prior to use.

The protective layer can comprise high-purity alginates, polyethylene glycols, cyclic and non-cyclic oligosaccharides and polysaccharides, antioxidative amino acids, proteins and vitamins. The protective layer preferably consists of a biocompatible high-viscosity polysaccharide which serves as a medium for added amino acids, proteins, vitamins and stabilizing polysaccharides. The high viscosity allows for rapid wettability of the surface. The attached protective layer adheres to the secondary coating and prevents the penetration of foreign substances during storage. In comparison with the specific ligands, the added amino acids, proteins and vitamins are present in increased concentrations and thereby able to attract or prevent the likelihood of damage to the target molecules by radical molecules or charge carriers and to reestablish chemical bonds destroyed by recombination processes.

Finished catheter 1 is packaged in a low-free environment. Final sterilization is carried out by use of gamma irradiation at a radiation dose of 25 kGy. Catheter 1 is intended for single use.

### Use of the Detection Device

Catheter 1 produced according to the invention with measuring structure 2, functional surface 10 and coupled detection receptors 12 is suitable for obtaining rare cells from the bloodstream. This includes the following examples of use:
obtaining cells, comprising preferably circulating endothelial cells or disseminated tumor cells, particularly of hematogenous metastasizing tumors e.g. with the humanized antibody anti-EpCAM which detects the cell surface protein EpCAM typical of many cancer cells,
obtaining and/or eliminating tumor markers or biomarkers, toxins, drugs, radioactive tracers and/or magnetic beads,
obtaining embryonic trophoblasts from the maternal blood circulation with e.g. specific antibody fragments (F (ab) fragments), and murine monoclonal antibodies (IgG) which can detect the cell surface protein HLA-G typical of trophoblast.

A possible application of detection device 1 is in prenatal and cancer diagnostics. Detection device 1 can, for example, be used for isolating fetal cells or tumor cells circulating in the bloodstream of pregnant women or cancer patients. For application, detection device 1 is introduced into the vein via a suitable, commercially available Braunüle cannula system and is applied into the venous blood circulation. The retention time in the vein can be about 30 minutes. After removal of detection device from the bloodstream, the cells bound on detection device 1 are further enriched by use of selective laboratory diagnostics and characterized by molecular or cell biology.

The aim of the minimally invasive procedure to be carried out is the selection of circulating endothelial cells, tumor cells or fetal cells from the blood. Due to the low cell concentration of the cells in the blood, a blood withdrawal of about 0.5 l would be needed to achieve the desired target cell number. This is however ruled out under medical aspects.

Circulating endothelial cells can be enriched with an antibody directed against CD-164, which is covalently bound to the hydrogel. Cancer tumor cells can be enriched with the EpCAM antibody (against the EpCAM antigen), which is humanized in its constant domains and is covalently bound to the hydrogel. Fetal trophoblasts can be enriched with an antibody from the mother's blood directed against HLA-G which is covalently bonded to the hydrogel.

Due to the arrangement of the measuring structure, the above-described use of the detection device can be simplified as it can be determined with only a small amount of time whether the required number of ligands or target molecules or target cells were bound or whether further extraction of sample material is required.

Detection device 1 described is suited for the enrichment or concentration of cells or biomolecules from body fluids or other liquid analytical samples. The liquid test samples are applied into detection device 1 or are immediately introduced by means of Luer-Lock withdrawing or sampling systems. The isolated and enriched material can immediately be supplied to a downstream diagnostic device, e.g. via lab-on-chip technology.

Detection device 1 is distinguished by a very simple handling owing to its design and can be used without any great expenditure of time by use of a kit in any diagnostic or clinical device or facility.

The invention claimed is:

1. A detection device for in vivo and/or in vitro enrichment of sample material, comprising a functional surface equipped with detection receptors, wherein said functional surface is arranged directly or indirectly on a measuring structure comprising optically conductive material for optical measurement of target molecules enriched on said detection receptors.

2. The detection device according to claim 1, wherein said measuring structure comprises an optical waveguide made of plastic material and/or wherein said measuring structure comprises an optical fiber.

3. The detection device according to claim 2, wherein the polymeric optical fiber comprises non-fluorescent material.

4. The detection device according to claim 2, wherein the polymeric optical fiber comprises amorphous fluoropolymer.

5. The detection device according to claim 2, wherein the polymeric optical fiber comprises fluororesin material.

6. The detection device according to claim 2, wherein the polymeric optical fiber comprises poly(methyl methacrylate).

7. The detection device according to claim 1, wherein said measuring structure comprises a single or a plurality of interconnected and/or twisted fibers and/or said measuring structure forms a continuous core and/or said measuring structure is formed as a carrier for said functional surface.

8. The detection device according to claim 1, wherein said measuring structure at its proximal end has a connection for an optical measuring instrument.

9. The detection device according to claim 8, wherein the connection is for a fiber-coupled spectrometer or a photodiode unit.

10. The detection device according to claim 1, wherein said measuring structure is at its proximal end section-wise enclosed by a holder.

11. The detection device according to claim 1, wherein said detection receptors comprise antibodies, antibody fragments, amino acid structures, nucleic acid structures and/or synthetic structures with a specific affinity to the surfaces of target cells.

12. The detection device according to claim 1, wherein: said functional surface extends over a functional section formed at the distal end of said detection device; said functional section preferably has a length of 20-60 mm and/or said functional section has a thickness of 0.1 to 1.0 mm, and/or wherein said functional surface is insertable into a vein or other vessels or body cavities.

13. The detection device according to claim 1, wherein said functional surface equipped with said detection receptors is formed by a functional layer comprising blood-repelling and/or biocompatible and/or hemocompatible material disposed along said functional section of said detection device, directly or indirectly on said measuring structure.

14. The detection device according to claim 1, wherein said functional surface is section-wise or entirely equipped with detection receptors, and/or said functional surface is equipped with detection receptors of different kinds or with detection receptors having different specificities.

15. The detection device according to claim 1, wherein target molecules enriched on said detection receptors can be marked by use of an antagonist.

16. The detection device according to claim 25, wherein the target molecules on said detection receptors are marked with a fluorescently marked antibody.

17. The detection device according to claim 1, wherein target molecules enriched on said detection receptors can be detectable by fluorescence spectroscopy, by use of said measuring structure to determine a wavelength shift and/or a change in intensity and/or a change in the luminescence decay.

18. The detection device according to claim 1, wherein said functional surface is coated with a protective layer, where said protective layer is soluble in body liquids; and/or said protective layer is biocompatible.

19. The detection device according to claim 1, wherein said detection receptors are operatively connected via linkers or organic functional groups to said functional surface to said measuring structure or said functional layer by chemical bonding, and/or said detection receptors are coupled via carboxyl groups to said functional surface, where coupling of said detection receptors is effected via carbodiimide chemistry and/or UV cross-linking, and/or wherein said carboxyl groups are provided at said measuring structure or within said functional layer.

20. The detection device according to claim 1, produced by:
a. providing a measuring structure with a functional section, and
b. functionalizing said functional section with detection receptors for forming a functional layer, where coupling said detection receptors is done by carbodiimide chemistry.

21. The detection device according to claim 1 for the invasive enrichment of sample material comprising circulating endothelial cells or disseminated tumor cells or for the elimination of drugs, for the elimination of radioactive tracers, for the elimination of magnetic beads, for obtaining tumor markers or biomarkers and/or for the elimination of toxins.

22. A method for the enrichment of sample material, comprising:
a. providing a detection device according to claim 1;
b. exposing said functional surface of said detection device to a sample liquid;
c. enriching sample material comprising cells, DNA, RNA, proteins, peptides, and/or synthetic molecules by exposing said sample material on said detection receptors of said detection device;
d. marking said sample material enriched on said detection receptors; and
e. optically detecting said sample material by said measuring structure by use of optic measurement.

23. The method according to claim 22 for the enrichment of sample material, wherein the sample material is marked with a fluorescently marked antibody.

24. The method according to claim 22 for the enrichment of sample material, wherein the sample material is optically detected by fluorescence measurement.
